(19) United States
(12) Patent Application Publication
(10) Pub. No.: US 2015/0167042 A1
(43) Pub. Date: Jun. 18, 2015

(54) Duckweed Hydrolysate and use Thereof

(71) Applicant: Lemnaceae Fermentation, Inc., Taoyuan County (TW)

(72) Inventor: Ming-Hsi Chiou, Taoyuan County (TW)

(21) Appl. No.: 14/635,673

(22) Filed: Mar. 2, 2015

Related U.S. Application Data
(62) Division of application No. 13/854,757, filed on Apr. 1, 2013.

(51) Int. Cl. C12P 23/00; C12P 5/02

(52) U.S. Cl. CPC C12P 23/00; C12P 5/026

(57) ABSTRACT

A duckweed hydrolysate is provided. A method for producing carotenoids comprising the incubation of microorganisms with the duckweed is also provided.

### CROSS-REFERENCE TO RELATED APPLICATIONS

This Divisional Patent Application claims priority to U.S. Ser. No. 13/854,757, filed Apr. 1, 2013, entitled “Duckweed Hydrolysate and use Thereof,” the contents of which is incorporated by reference herein in its entirety.

### FIELD OF THE INVENTION

The invention relates to a hydrolysate product of duckweed, which can be used as a nutrient for cultivating microorganisms, such as E. coli, to increase the yields of cell biomass and cell products.

### BACKGROUND OF THE INVENTION

Duckweed, comprising plants of the Lemnaceae family, is known to have fast doubling time and has been used as feed for ducks, chickens, fishes and shrimps. Duckweed can also be used to treat wastewater, and to convert carbon dioxide to biomass. Therefore, duckweed is considered potentially useful to solve critical water and climate problems. Cheng et al. (Growing Duckweed to recover nutrients from wastewaters and for production of fuel ethanol and animal feed. Clean. 37 (1). 17-26 (2009)) discloses that duckweed can remove nitrogen and phosphorus content from the wastewater and that duckweed biomass can be used as an alternative source of fermentable carbohydrate for fuel ethanol production.

Microorganisms (either naturally occurring or recombinated) have been widely used to produce polypeptides, such as pharmaceutically active proteins, and secondary metabolites, such as vitamins and carotenoids. However, attempts to produce such products using microorganisms have suffered many problems. For example, the costs of the components of the media are high and the production rate of large scale fermentation may be poor. U.S. Pat. No. 7,122,341 B1 (Liao, 2000) discloses a method for the engineering of metabolic control. The method includes obtaining an E. coli strain that has a higher lycopene yield.

However, it is still necessary to develop an inexpensive way to improve large scale fermentation yield.

### SUMMARY OF THE INVENTION

The present invention aims to provide an inexpensive supplement for microorganism fermentation to increase the yields of cell biomass and cell products.

The present invention therefore provides a duckweed hydrolysate, which is obtained by the process comprising the steps of:
(a) obtaining duckweed juice from duckweed;
(b) hydrolyzing the duckweed juice with one or more proteases at a temperature of about 25° C. to about 75° C. for about 6 to about 48 hours; and
(c) collecting the duckweed hydrolysate.

Also disclosed herein is a method for producing a carotenoid comprising the steps of: incubating a carotenoid producing microorganism in a medium comprising the duckweed hydrolysate of the present invention under suitable conditions; harvesting the cells; and isolating the carotenoid from the cells.

### DETAILED DESCRIPTION OF THE INVENTION

The present invention can be understood more readily by reference to the following detailed description of various embodiments of the invention, the examples, and the tables with their relevant descriptions. Unless otherwise defined, all terms (including technical and scientific terms) used herein have the same meaning as commonly understood by one of ordinary skill in the art to which this invention belongs. It will be further understood that terms such as those defined in commonly used dictionaries should be interpreted consistently with their meaning in the context of the relevant art and will not be interpreted in an idealized or overly formal sense unless expressly so defined herein. It is also to be understood that the terminology used herein is for the purpose of describing particular embodiments only and is not intended to be limiting.

It must be noted that, as used in the specification and the appended claims, the singular forms “a,” “an” and “the” include plural referents unless the context clearly dictates otherwise. Thus, unless otherwise required by context, singular terms shall include the plural and plural terms shall include the singular.

Often, ranges are expressed herein as from “about” one particular value and/or to “about” another particular value. When such a range is expressed, an embodiment includes the range from the one particular value and/or to the other particular value. Similarly, when values are expressed as approximations, by use of the word “about,” it will be understood that the particular value forms another embodiment. It will be further understood that the endpoints of each of the ranges are significant both in relation to and independently of the other endpoint. As used herein the term “about” refers to ±10%.

The present invention provides a duckweed hydrolysate, which is obtained by the process comprising the steps of:
(a) obtaining duckweed juice from duckweed;
(b) hydrolyzing the duckweed juice with one or more proteases at a temperature of about 25° C. to about 75° C. for about 6 to about 48 hours; and
(c) collecting the duckweed hydrolysate.

The term “duckweed” as used herein denotes a plant of the Lemnaceae family, which includes the genera of Lemna, Spirodela, Wolffia, and Wolfiella. The duckweed used in the present invention may be, but not limited to, Lemna aequinoctialis, Lemna gibba, Lemna disperma, Lemna minor, Lemna ecuadoriensis, Lemna japonica, Lemna obscura, Spirodela punctata, Spirodela polyrrhiza, and Wolffia arrhiza.

The term “duckweed juice” refers to the liquid portion of the duckweed. However, the liquid portion does not exclude small particles from the duckweed, for example, particles less than about 0.25 mm.

According to the invention, duckweed juice can be obtained by any methods known in the art. For example, duckweed can be ground by a grinder or treated with a juicer, after which the lipid part is subjected to filtration and/or centrifugation to separate the duckweed juice from the solid residue.

The term “protease” refers to any products or enzymes derived from microorganisms, animals or plants that can hydrolyze proteins and polypeptides so that they become peptides, oligopeptides and/or amino acids. The protease of the invention can be endoproteases, exopeptidases or combinations thereof The protease can be used in the invention includes, but is not limited to, thermolysin, pepsin, trypsin, bromelain, Alcalase (Novozyme), Flavorzyme, Esperase, PTN 6.0 S, Acid Protease, Protamex, Protease A, Protease M, Protease N, Protease NL, Protease P, Protease S, Protin SD, Thermoase, Flavorpro and Promod. Preferably, the protease is Protamex.

According to the invention, the one or more proteases can be added to the duckweed juice together, sequentially or separately, and the amounts of proteases added to the hydrolysis reaction depend on the species of proteases used. For example, the one or more proteases can be in an amount of about 1 A.U. to about 150 A.U. per liter of the duckweed juice. Preferably, the one or more proteases are in an amount of about 2 A.U. to about 50 A.U. More preferably, the one or more proteases are in an amount of about 3 A.U. to about 15 A.

U. According to the invention, the temperature and time of the hydrolysis reaction depend on the species of protease used. The temperature of the hydrolysis reaction may range from about 25° C. to about 75° C., preferably from about 35° C. to about 60° C., and more preferably from about 35° C. to about 45° C. The time of the hydrolysis reaction may range from about 6 to about 48 hours, preferably from about 12 to about 36 hours, and more preferably from about 12 to about 24 hours. Optionally, the protease activity can be inactivated by any methods known in the art at the end of the hydrolysis reaction.

According to the invention, the process further comprises step (d) where the duckweed hydrolysate collected from step (c) is concentrated and/or dried so that a concentrated and/or dried duckweed hydrolysate is obtained. The concentrating and drying methods are known in the art and include, but are not limited to, heating, freeze drying, spray drying, drum-dryer drying, fluid-bed drying, and any combinations thereof.

The present invention also provides a method for producing a carotenoid comprising the steps of: incubating a carotenoid producing microorganism in a medium comprising an effective amount of the duckweed hydrolysate of the invention at suitable conditions; harvesting the cells; and isolating the carotenoid from the cells.

The term “carotenoid” represents any of various usually yellow to red pigments found widely in microorganisms, plants and animals and characterized chemically by a long aliphatic polyene (C40) chain composed of eight isoprene units. Examples of carotenoids include β-carotene, zeaxanthin, canthaxanthin, β-cryptoxanthin, astaxanthin, lycopene, and lutein.

The term “carotenoid producing microorganism” represents any microorganisms that can produce carotenoids. The microorganisms may be any host cells transformed by one or more exogenous genes, which encode the enzymes involving carotenoid synthesis, and can express the enzymes and synthesize carotenoids at suitable conditions. The host cells include, but are not limited to, Escherichia coli, Saccharomyces cerevisiae, Blakeslea trispora, Agrobacterium aurantiacum, Haematococcus pluvialis, and Xanthophyllomyces dendrorhous. Preferably, the host cell is Escherichia coli. The microorganisms can be constructed by any conventional materials and technologies known in the art, such as those disclosed in U.S. Pat. No. 7,122,341 B1 U.S. Pat. No. 5,429,935, Misawa et al. (Elucidation of the Erwinia uredovora Carotenoid Biosynthetic Pathway by Functional Analysis of Gene Products Expressed in Escherichia coli; Journal of Bacteriology, Vol. 172, No, 12, pp. 6704-6712 (1990)), Wang et al. (Engineered Isoprenoid Pathway Enhances Astrxanthin Production in Escherichia coli; Biotechnology and Bioengineering, Vol. 62, No. 2, pp. 235-241 (1999)), Farmer et al. (Improving lycopene production in Escherichia coli by engineering metabolic control; Nature Biotechnology, Vo. 18, pp. 533-537 (2000)), Scaife et al. (Characterization of Cyanobacterial β-Carotene Ketolase and Hydrolase Genes in Escherichia coli, and Their Application for Astaxanthin Biosynthesis; Biotechnology and Bioengineering, Vol. 103, No. 5, pp. 944-955 (2009)), and Scaife et al. (Comparative Analysis of β-Carotene Hydrolase Genes for Astaxanthin Biosynthesis; J. Nat. Prod., 75, 1117-1124 (2012)). The whole references are incorporated herein as a part of the specification.

The “suitable conditions” for incubating the microorganism means that the conditions, e.g., temperature and incubation time, allow the microorganism to grow, propagate, express the enzymes relating to carotenoid synthesis, and synthesize carotenoids. The exact conditions required will vary with the species of the microorganism and the types of incubation. According to the invention, the incubation may be any type of fermentation known in the art, such as batch fermentation, fed batch fermentation and continuous fermentation.

The “medium comprising an effective amount of the duckweed hydrolysate” used in the invention may include, in addition to the duckweed hydrolysate, any components known for incubating microorganisms, such as nitrogen sources, e.g., yeast extract, peptone and amino acids; carbon sources, e.g., glucose and glycerol; salts, e.g., potassium salts and magnesium salts; and a buffer, e.g., phosphate buffer. The term “effective amount” means that the amount of duckweed hydrolysate can effectively increase the yields of cell biomass and carotenoids.

The methods for harvesting cells and for isolating carotenoids from cells are all known in the art, such as those disclosed in EP 2 088 199 A1. The whole references are incorporated herein as a part of the specification.

### EXAMPLES

### Example 1

### Preparation of Duckweed Hydrolysate

100 kg of fresh duckweed (Lemna aequinoctialis) was washed and ground by a grinder or treated with a juicer. The liquid portion was collected and filtered to obtain the duckweed juice. The obtained duckweed juice (60 L) was treated with 450 A.U. of

Protamex (purchased from Novozymes) at 40° C. for 20 hours to obtain the duckweed hydrolysate.

### Example 2

### Production of Lycopene without Duckweed Hydrolysate

### (1) Microorganism:

E. coli CCRC 940321, which produces lycopene, was constructed based on the methods disclosed in U.S. Pat. No. 7,122,341 B1. Briefly, plasmids pCW9 and p2IDI were introduced into E. coli JCL1613 to obtain the lycopene producing host cell. E. coli CCRC 940321 can be obtained from the Food Industry Research and Development Institute (No. 331, Shihpin Rd., Hsinchu City, Taiwan, R.O.C.).

### (2) Media:

Seed Medium:

| | |
|---|---|
| 2% (w/v) | yeast extract (Difico) |
| 2% (w/v) | tryptone (Difico) |
| 2% (w/v) | glycerol (Sigma) |

Batch Medium: (2 L)

| | |
|---|---|
| 3.6% (w/v) | yeast extract (Difico) |
| 0.54% (w/v) | dipotassium hydrogen phosphate (J. T. Baker) |
| 1.07% (w/v) | monopotassium dihydrogen phosphate (J. T. Baker) |
| 1% (w/v) | glycerol (Sigma) |

Feed Medium: (1 L)

| | |
|---|---|
| 10% (w/v) | monosodium glutamate |
| 1.3% (w/v) | yeast extract (Difico) |
| 2.5% (w/v) | amino acid mixture (Sigma) including: 20% (w/w) alanine, |
| | 10% (w/w) arginine, 20% (w/w) aspartic acid, 20% (w/w) |
| | glycine, 10% (w/w) methionine, and 20% (w/w) lysine |
| 1% (w/v) | magnesium sulfate (J. T. Baker) |
| 60% (w/v) | glycerol (Sigma) |

### (3) Method:

Frozen E. coli CCRC 940321 was introduced into a 250 mL flask containing 50 mL of the seed medium, and incubated at 32° C. with shaking at 150 rpm for 12 hours. The activated cells were transferred to a 5 L-fermentor containing 2 L of the batch medium and incubated at 30° C. with stirring at 400 to 600 rpm and aerating at 2 L/min. When the cell density (OD₆₀₀) became between 15 and 25, the feed medium was added to the fermentor (pH was controlled at 6.8-7.0). 108 hours later, the cell density (OD₆₀₀) reached 140 (corresponding to 46.2 g of dry cells per liter of the total culture) and the cells were harvested. 16 mg of lycopene was obtained from a gram of dry cells.

### Example 3

### Production of Lycopene with Duckweed Hydrolysate

### (1) Microorganism:

E. coli CCRC 940321

### (2) Media:

Seed Medium:

| | |
|---|---|
| 2% (w/v) | yeast extract (Difico) |
| 2% (w/v) | tryptone (Difico) |
| 2% (w/v) | glycerol (Sigma) |

Batch Medium: (2 L)

| | | |
|---|---|---|
| 3.6% | (w/v) | yeast extract (Difico) |
| 0.54% | (w/v) | dipotassium hydrogen phosphate (J. T. Baker) |
| 1.07% | (w/v) | monopotassium dihydrogen phosphate (J. T. Baker) |
| 1% | (w/v) | glycerol (Sigma) |
| 20 | mL | duckweed hydrolysate obtained from Example 1 |

Feed Medium: (1 L)

| | | |
|---|---|---|
| 10% | (w/v) | monosodium glutamate |
| 1.3% | (w/v) | yeast extract (Difico) |
| 2.5% | (w/v) | amino acid mixture (Sigma): 20% (w/w) alanine, 10% |
| | | (w/w) arginine, 20% (w/w) aspartic acid, 20% (w/w) |
| | | glycine, 10% (w/w) methionine, and 20% (w/w) lysine |
| 1% | (w/v) | magnesium sulfate (J. T. Baker) |
| 60% | (w/v) | glycerol (Sigma) |
| 330 | mL | duckweed hydrolysate obtained from Example 1 |

### (3) Method:

Frozen E. coli CCRC 940321 was introduced into a 250 mL flask containing 50 mL of the seed medium, and incubated at 32° C. with shaking at 150 rpm for 12 hours. The activated cells were transferred to a 5 L-fermentor containing 2 L of the batch medium and incubated at 30° C. with stirring at 400 to 600 rpm and aerating at 2 L/min. When the cell density (OD₆₀₀) became between 15 and 25, the feed medium was added to the fermentor (pH was controlled at 6.8-7.0). 108 hours later, the cell density (OD₆₀₀) reached 190 (corresponding to 62.7 g of dry cells per liter of the total culture) and the cells were harvested. 21 mg of lycopene was obtained from a gram of dry cells.

### Example 4

### Production of β-Carotene without Duckweed Hydrolysate

### (1) Microorganism:

β-carotene producing cells were constructed based on the method disclosed in Misawa et al. (1990). Briefly, crtY gene (encoding lycopene cyclase) was amplified from Erwinia uredovora (ATCC19321) by PCR, the PCR fragment was cloned into the SacI site of plasmid pCW9 to obtain plasmid pCW9Y, and plasmids pCW9Y and p2IDI were then introduced into E. coli JCL1613 to obtain the β-carotene producing host cell.

### (2) Media:

The same as those of Example 2.

### (3) Method:

Frozen β-carotene producing cells were introduced into a 250 mL flask containing 50 mL of the seed medium, and incubated at 32° C. with shaking at 150 rpm for 12 hours. The activated cells were transferred to a 5 L-fermentor containing 2 L of the batch medium and incubated at 30° C. with stirring at 400 to 600 rpm and aerating at 2 L/min. When the cell density (OD₆₀₀) became between 15 and 25, the feed medium was added to the fermentor (pH was controlled at 6.8-7.0). 132 hours later, the cell density (OD₆₀₀) reached 135 (corresponding to 44.6 g of dry cells per liter of the total culture) and the cells were harvested. 15 mg of β-carotene was obtained from a gram of dry cells.

### Example 5

### Production of β-Carotene with Duckweed Hydrolysate

### (1) Microorganism:

The same as that constructed in Example 4.

### (2) Media:

The same as those of Example 3.

### (3) Method:

Frozen β-carotene producing cells were introduced into a 250 mL flask containing 50 mL of the seed medium, and incubated at 32° C. with shaking at 150 rpm for 12 hours. The activated cells were transferred to a 5 L-fermentor containing 2 L of the batch medium and incubated at 30° C. with stirring at 400 to 600 rpm and aerating at 2 L/min. When the cell density (OD₆₀₀) became between 15 and 25, the feed medium was added to the fermentor (pH was controlled at 6.8-7.0). 132 hours later, the cell density (OD₆₀₀) reached 185 (corresponding to 61.1 g of dry cells per liter of the total culture) and the cells were harvested. 15 mg of β-carotene was obtained from a gram of dry cells.

### Example 6

### Production of Zeaxanthin without Duckweed Hydrolysate

### (1) Microorganism:

Zeaxanthin producing cells were constructed based on the method disclosed in Misawa et al. (1990). Briefly, crtZ gene (encoding β-carotene hydroxylase) was amplified from Erwinia uredovora (ATCC19321) by PCR, the PCR fragment was cloned into the Apal site of plasmid pCW9Y to obtain plasmid pCW9YZ, and plasmids pCW9YZ and p2IDI were then introduced into E. coli JCL1613 to obtain the zeaxanthin producing host cell.

### (2) Media:

The same as those of Example 2.

### (3) Method:

Frozen zeaxanthin producing cells were introduced into a 250 mL flask containing 50 mL of the seed medium, and incubated at 32° C. with shaking at 150 rpm for 12 hours. The activated cells were transferred to a 5 L-fermentor containing 2 L of the batch medium and incubated at 30° C. with stirring at 400 to 600 rpm and aerating at 2 L/min. When the cell density (OD₆₀₀) became between 15 and 25, the feed medium was added to the fermentor (pH was controlled at 6.8-7.0). 120 hours later, the cell density (OD₆₀₀) reached 122 (corresponding to 40.3 g of dry cells per liter of the total culture) and the cells were harvested. 10 mg of β-carotene was obtained from a gram of dry cells.

### Example 7

### Production of Zeaxanthin with Duckweed Hydrolysate

### (1) Microorganism:

The same as that constructed in Example 4.

### (2) Media:

The same as those of Example 3.

### (3) Method:

Frozen zeaxanthin producing cells were introduced into a 250 mL flask containing 50 mL of the seed medium, and incubated at 32° C. with shaking at 150 rpm for 12 hours. The activated cells were transferred to a 5 L-fermentor containing 2 L of the batch medium and incubated at 30° C. with stirring at 400 to 600 rpm and aerating at 2 L/min. When the cell density (OD₆₀₀) became between 15 and 25, the feed medium was added to the fermentor (pH was controlled at 6.8-7.0). 132 hours later, the cell density (OD₆₀₀) reached 175 (corresponding to 57.8 g of dry cells per liter of the total culture) and the cells were harvested. 13 mg of β-carotene was obtained from a gram of dry cells.

### Example 8

### Production of Canthaxanthin without Duckweed Hydrolysate

### (1) Microorganism:

Canthaxanthin producing cells were constructed based on the methods disclosed in Misawa et al. (1990) and Misawa et al. (Structure and Functional Analysis of a Marine Bacterial Carotenoid Biosynthesis Gene Cluster and Astaxanthin Biosynthetic Pathway Proposed at the Gene Level; Journal of Bacteriology, Vol. 177, No. 22, pp. 6575-6584 (1995)) Briefly, crtW gene (encoding β-carotene ketolase) was amplified from Brevundimonas aurantiaca (ATCC15266) by PCR, the PCR fragment was cloned into the ApaI site of plasmid pCW9Y to obtain plasmid pCW9YW, and plasmids pCW9YW and p2IDI were then introduced into E. coli JCL1613 to obtain the canthaxanthin producing host cell.

### (2) Media:

The same as those of Example 2.

### (3) Method:

Frozen canthaxanthin producing cells were introduced into a 250 mL flask containing 50 mL of the seed medium, and incubated at 32° C. with shaking at 150 rpm for 12 hours. The activated cells were transferred to a 5 L-fermentor containing 2 L of the batch medium and incubated at 30° C. with stirring at 400 to 600 rpm and aerating at 2 L/min. When the cell density (OD₆₀₀) became between 15 and 25, the feed medium was added to the fermentor (pH was controlled at 6.8-7.0). 124 hours later, the cell density (OD₆₀₀) reached 142 (corresponding to 46.9 g of dry cells per liter of the total culture) and the cells were harvested. 14 mg of β-carotene was obtained from a gram of dry cells.

### Example 9

### Production of Canthaxanthin with Duckweed Hydrolysate

### (1) Microorganism:

The same as that constructed in Example 8.

### (2) Media:

The same as those of Example 3.

### (3) Method:

Frozen canthaxanthin producing cells were introduced into a 250 mL flask containing 50 mL of the seed medium, and incubated at 32° C. with shaking at 150 rpm for 12 hours. The activated cells were transferred to a 5 L-fermentor containing 2 L of the batch medium and incubated at 30° C. with stirring at 400 to 600 rpm and aerating at 2 L/min. When the cell density (OD₆₀₀) became between 15 and 25, the feed medium was added to the fermentor (pH was controlled at 6.8-7.0). 124 hours later, the cell density (OD₆₀₀) reached 180 (corresponding to 59.4 g of dry cells per liter of the total culture) and the cells were harvested. 18 mg of β-carotene was obtained from a gram of dry cells.

The incubation conditions and results of Examples 2 to 9 are summarized in Table 1 below.

TABLE 1

| | Bacteria Strains | | | |
|---|---|---|---|---|
| | Lycopene | β-Carotene | Zeaxanthin | Canthaxanthin |
| | producing | producing | producing | producing |
| Results | E. coli | E. coli | E. coli | E. coli |
| No duckweed hydrolysate: | Example 2 | Example 4 | Example 6 | Example 8 |
| Biomass (g/L) | 46.2 g/L | 44.6 g/L | 40.3 g/L | 46.9 g/L |
| Production rate (mg/g) | 16 mg/g | 15 mg/g | 10 mg/g | 14 mg/g |
| With duckweed hydrolysate: | Example 3 | Example 5 | Example 7 | Example 8 |
| Biomass (g/L) | 62.7 g/L | 61.1 g/L | 57.8 g/L | 59.4 g/L |
| Production rate (mg/g) | 21 mg/g | 22 mg/g | 13 mg/g | 18 mg/g |

As shown in Table 1, the addition of duckweed hydrolysate can significantly increase not only the biomass of the cell but also the production rates of carotenoids.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1.-9. (canceled)

10. A method for producing a carotenoid comprising the steps of:
incubating a carotenoid producing microorganism in a medium comprising an effective amount of a duckweed hydrolysate at suitable conditions;
harvesting the cells; and
isolating the carotenoid from the cells.

11. The method according to claim 10, wherein the carotenoid is selected from the group consisting of β-carotene, zeaxanthin, canthaxanthin, β-cryptoxanthin, astaxanthin, lycopene, and lutein.

12. The method according to claim 10, wherein the carotenoid producing microorganism is selected from the group consisting of Escherichia coli, Saccharomyces cerevisiae, Blakeslea trispora, Agrobacterium aurantiacum, Haematococcus pluvialis, and Xanthophyllomyces dendrorhous.

13. The method according to claim 12, wherein the carotenoid producing microorganism is E. coli.

14.-17. (canceled)

18. The method according to claim 10, wherein the duckweed hydrolysate is obtained by the process comprising the steps of: (a) obtaining duckweed juice from duckweed; (b) hydrolyzing the duckweed juice with one or more proteases at a temperature of about 25° C. to about 75° C. for about 6 to about 48 hours; and (c) collecting the duckweed hydrolysate.
